(19) United States
(12) Patent Application Publication
(10) Pub. No.: US 2003/0003584 A1
(43) Pub. Date: Jan. 2, 2003

(54) Liposomal vector binding protein for hepatocyte DNA delivery

(76) Inventor: Darius John Adams, Wayne, NJ (US)

Correspondence Address: DARIUS JOHN ADAMS, 15 HOLMES LANE, WAYNE, NJ, 07470, US

(21) Appl. No.: 09/681,938

(22) Filed: Jun. 28, 2001

(51) Int. Cl. A61K 48/00; A61K 9/127; C12N 15/88

(52) U.S. Cl. 435/458; 424/450; 514/44

(57) ABSTRACT

Liposomes have been shown to be very safe complexes with virtually no side effects. Utilizing liposomes for gene therapy has been hampered by their non-specific nature of gene delivery. This protein sequence contains a DNA binding region, an alpha helix region which will transverse the liposome wall, and a receptor binding region to allow for targeting to a specific cell type, in this case, a hepatocyte.

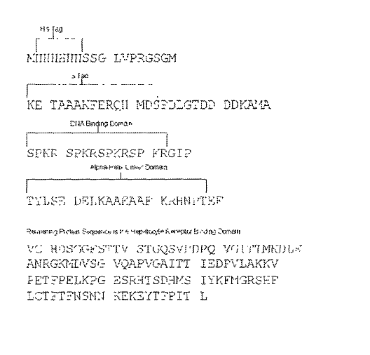

### BRIEF DESCRIPTION OF SEQUENCES

Protein Sequence: MHHHHHHSSG LVPRGSGMKE TAAAKFERQH MDSPDLGTDD DDKAMASPKR SPKRSPKRSP KRGIPTYLSE DELKAAEAAF KRHNPTEFVG HDSGGFSTTV STGQSVPDPQ VGITTMKDLK ANRGKMDVSG VQAPVGAITT IEDPVLAKKV PETFPELKPG ESRHTSDHMS IYKFMGRSHF LCTFTFNSNN KEKEYTFPIT L

### DETAILED DESCRIPTION

Liposomes have been found to be safe vectors, however, they lack specificity. This binding protein will be very useful in guiding liposomes to hepatocytes for DNA delivery into this specific cell subtype. The invention would be a particularly useful in gene therapy.

This invention is a protein sequence that contains a DNA binding domain, a lipophilic alpha chain, and a hepatocyte receptor binding region.

1. a method of directing or targeting liposomes to hepatocytes while securing DNA for delivery.
